(19) United States
(12) Patent Application Publication
(10) Pub. No.: US 2019/0282127 A1
(43) Pub. Date: Sep. 19, 2019

(54) SYSTEM AND METHOD FOR EARLY DETECTION OF TRANSIENT ISCHEMIC ATTACK

(71) Applicant: KONINKLIJKE PHILIPS N.V., EINDHOVEN (NL)

(72) Inventors: MARIANA NIKOLOVA-SIMONS, BOLTON, MA (US); TINE SMITS, BEERSE (BE); JORN OP DEN BUIJS, EINDHOVEN (NL); MARTEN JEROEN PIJL, EINDHOVEN (NL); JOSEF HERIBERT BALDUS, AACHEN (DE); LINDA SCHERTZER, FRAMINGHAM, MA (US)

(21) Appl. No.: 16/301,643

(22) PCT Filed: May 23, 2017
(86) PCT No.: PCT/EP2017/062399

§ 371 Date: Nov. 14, 2018

Related U.S. Application Data
(60) Provisional application No. 62/339,997, filed on May 23, 2016.

(51) Int. Cl. A61B 5/11; A61B 5/00

(52) U.S. Cl. CPC A61B 5/112; A61B 5/0022; A61B 5/1124; G16H 40/67; A61B 5/1117; A61B 5/4064; A61B 5/749; A61B 5/746

(57) ABSTRACT

In a transient ischemic attack (TIA) alerting device, a motion sensor (24) is configured to attach to or be worn by a subject. A wearable electronic data processing device (10) includes a speaker (14), a microphone (16), a radio transmitter (26), and an electronic processor (40). The processor is programmed to perform a TIA alerting method including: detecting a TIA-symptomatic gait type by processing subject motion data acquired by the motion sensor; responsive to detecting the TIA-symptomatic gait type, executing a TIA assessment questionnaire (70) by outputting questions using the speaker and receiving spoken responses using the microphone; determining a TIA assessment based at least on the spoken responses to the questions of the TIA assessment questionnaire; and conditional on the TIA assessment indicating a possible TIA, outputting a TIA alert radio signal using the radio transmitter.

### FIELD

The following relates generally to the patient monitoring arts, cardiovascular disease prevention arts, cardiovascular disease diagnostic arts, patient wellness arts, and related arts.

### BACKGROUND

Transient ischemic attack (TIA) is an episode of impaired brain function due to loss of blood flow to the brain or a critical neural region. Sometimes referred to as a “mini-stroke”, a TIA differs from a stroke in that the TIA generally does not produce lasting neurological damage or widespread tissue death. Symptoms of a TIA episode typically last a few minutes, and rarely last longer than 24 hours. However, TIAs are nonetheless serious events because a TIA episode implies a problem with blood flow to the affected brain or neural tissue. TIA episodes are recognized risk factors for stroke or vascular dementia, and TIA episodes are also often observed in connection with non-vascular dementia types such as Alzheimer's disease. Early detection of TIA can thus lead to early detection of an elevated risk of stroke or of incipient dementia.

The following discloses new and improved systems and methods that address the above referenced issues, and others.

### SUMMARY

In one disclosed aspect, a transient ischemic attack (TIA) alerting device comprises a motion sensor configured to attach to or be worn by a subject, and a wearable electronic data processing device including a speaker, a microphone, a radio transmitter, and an electronic processor. The processor is programmed to perform a TIA alerting method including: detecting a TIA-symptomatic gait type by processing subject motion data acquired by the motion sensor; responsive to detecting the TIA-symptomatic gait type, executing a TIA assessment questionnaire by outputting questions using the speaker and receiving spoken responses using the microphone; determining a TIA assessment based at least on the spoken responses to the questions of the TIA assessment questionnaire; and conditional on the TIA assessment indicating a possible TIA, outputting a TIA alert radio signal using the radio transmitter. In some embodiments, the TIA-symptomatic gait type includes an ataxic gait detected based on stride length and stride regularity features generated from the motion sensor data. In some embodiments, the TIA-symptomatic gait type includes a hemiplegic gait detected based on gait symmetry features generated from the motion sensor data. In some embodiments, the TIA-symptomatic gait type includes a sensory gait detected based on features generated from flat-foot and heel/toe off stride phase portions of the motion sensor data. In some embodiments the motion sensor comprises an accelerometer, although additional or other types of motion sensors such as a gyroscope, pressure transducer, magnetometer, or so forth may be employed. Some embodiments further include a base station configured to receive the TIA alert radio signal and to generate a telephonic emergency call in response to receiving the TIA alert radio signal. In other embodiments, the radio transmitter comprises a cellular telephone transmitter and the TIA alert radio signal comprises a cellular telephone call. The motion sensor may be integrated with the wearable electronic data processing device, or they may be separate components with the motion sensor positioned optimally to detect TIA-symptomatic gait type(s). In some embodiments the wearable electronic data processing device further includes an electronic data storage, and the electronic processor is programmed to store at least one spoken response to at least one question of the TIA assessment questionnaire in the electronic data storage.

In another disclosed aspect, a TIA alerting device comprises a physiological sensor configured to attach to or be worn by a subject; and an electronic data processing device including a speaker, a microphone, and an electronic processor programmed to perform a TIA alerting method including: detecting a TIA symptom by processing physiological data acquired by the physiological sensor; responsive to detecting the TIA symptom, executing a TIA assessment questionnaire by outputting questions using the speaker and receiving spoken responses using the microphone; determining a TIA assessment based at least on the spoken responses to the questions of the TIA assessment questionnaire; and conditional on the TIA assessment indicating a possible TIA, outputting a TIA alert signal.

In another disclosed aspect, a TIA alerting method comprises: detecting a TIA-symptomatic gait type by processing subject motion data acquired by a motion sensor; responsive to detecting the TIA-symptomatic gait type, executing a TIA assessment questionnaire by electronically outputting questions using a speaker and electronically receiving spoken responses using a microphone; determining a TIA assessment based at least on the electronically received spoken responses to the electronically outputted questions of the TIA assessment questionnaire; and conditional on the TIA assessment indicating a possible TIA, outputting a TIA alert radio signal using a radio transmitter. In some embodiments the detecting comprises detecting an ataxic gait based on stride length and stride regularity extracted from the subject motion data, a hemiplegic gait detected based on gait symmetry extracted from the subject motion data, or a sensory gait detected based on flat-foot and heel/toe off stride phases extracted from the subject motion data.

One advantage resides in providing for early detection of transient ischemic attack (TIA) episodes.

Another advantage resides in providing for early detection of predisposition to stroke by way of early detection of TIA episodes.

Another advantage resides in providing for early detection of incipient dementia by way of early detection of TIA episodes.

Another advantage resides in providing the foregoing benefits by way of non-invasive patient monitoring.

Another advantage resides in providing the foregoing benefits by way of continuous in-residence patient monitoring.

Another advantage resides in providing the foregoing benefits including differentiation of a TIA episode from other conditions having similar symptoms.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically shows a transient ischemic attack (TIA) alerting device.

FIG. 2 diagrammatically shows a TIA alerting method suitably performed using the TIA alerting device of FIG. 1.

### DETAILED DESCRIPTION

As previously mentioned, a TIA episode implies a problem with blood flow to the affected brain or neural tissue, TIA episodes are recognized risk factors for stroke or vascular dementia as well as being associated with non-vascular dementia types such as Alzheimer's disease. For these and other reasons, early detection of TIA is beneficial.

Because TIA symptoms are transient, it can be difficult to diagnose a TIA more than 24 hours or so after occurrence of the TIA. Further, if the TIA episode occurs when the victim is alone, then there is no one to observe the symptoms and the victim may be in an impaired cognitive state due to the effects of the TIA. A further complication is differential diagnoses: other conditions such as certain types of migraines, electrolyte abnormalities, or alcoholic inebriation can lead to symptoms closely mimicking those of a TIA. Accordingly, a TIA can be most effectively detected immediately after onset of the TIA episode, while the transient TIA symptoms are most prevalent.

In practice, however, TIA diagnoses are commonly made long after occurrence of the TIA episode, i.e. after the transient symptoms have disappeared. For example, diagnosis may be made by a doctor during a later office visit. Such TIA diagnosis relies upon the patient bringing up the episode with the doctor, or the doctor having the insight based on discussion with the patient, physical examination, or laboratory test results to ask whether such an episode has occurred. Diagnosis in a doctor's office visit setting is also reliant upon the patient's accuracy in recalling the symptoms, such accuracy being potentially impaired by chronic conditions of the patient (e.g. dementia) and/or by the effects of the TIA itself. If TIA episodes are not diagnosed, the underlying vascular conditions are likely to persist and eventually manifest in other, more serious ways, such as in a stroke or onset of vascular dementia.

Disclosed herein are TIA alerting devices that address the foregoing problems and others. In some disclosed embodiments, the TIA alerting device employs a two-level detection process. First, a physical symptom is detected that indicates a possible TIA episode. The detection triggers the second level in which a TIA assessment questionnaire is presented. If the combination of the detected physical symptom and the responses to the questionnaire indicate a possible TIA, then appropriate follow-up action is taken, such as placing a call to “911” or another emergency response telephone number to summon an ambulance, and/or placing a call to a personal emergency response system (PERS) call center. The approach thereby enables posing appropriate questions to detect a possible TIA, but doing so in a timely fashion, i.e. after automated detection of a physical symptom that suggests a TIA may have just occurred.

In illustrative embodiments described herein, the detected physical symptom is a gait type that is indicative of TIA, such as an ataxic gait (characterized by unsteadiness or lack of coordination), a hemiplegic gait (characterized by strong left-right asymmetry and/or circumduction), or a sensory gait (also called “stomping gait”, characterized by heavy footfalls due to compromised propioreception in the foot).

The disclosed TIA alerting devices are not intended to provide a clinical diagnosis of a TIA, but rather are intended to provide an early alert of a possible TIA that should trigger rapid follow-up examination by a doctor or other care professional to make an appropriate medical diagnosis. However, in recognition that TIA symptoms are generally transitory in nature, some TIA alerting device embodiments disclosed herein collect patient data during the second level (execution of the questionnaire) that may be usefully reviewed by the doctor in making the diagnosis. For example, spoken responses to the questionnaire may be recorded using a microphone, and these recorded responses may be reviewed by the doctor to detect slurred speech which is a common TIA symptom. Advantageously, the responses are recorded within a few seconds to a few minutes after detection of the TIA-suggestive gait type (or other detected TIA-suggestive symptom) so that the recorded responses are likely to capture slurred speech (if present due to a TIA) while this transient symptom is present.

In examples described herein, the term “client” is used to denote the person monitored by the TIA alerting device. That is, in a typical operational scenario the “client” experiences a TIA episode which is detected by the TIA alerting device, and the “client” provides the responses to the subsequently presented questionnaire (assuming the client is capable of doing so if the client is unresponsive due to severe TIA or stroke or other debilitating condition then a TIA alert or other emergency protocol is performed). The term “client” reflects a typical contemplated commercial implementation in which the TIA alerting device is provided by a Personal Emergency Response Service (PERS). In some existing commercial PERS services, a user wears a personal help button (PHB) device that emits a radio signal that is detected by a base station located in the client's home. The base station includes a speakerphone, and the radio signal from the PHB device triggers the base station to call a PERS call center so as to place the subscriber into telephonic contact with a call center agent via the speakerphone of the base station. In another design, the PHB device includes an on-board cellular transceiver, e.g. operating using a 2G, 3G, or 4G connection, and the PHB device directly places the call to the PERS agent via cellular connection and using a microphone/speaker assembly built into the PHB device. In yet another approach, the PHB device may pair with a cellular telephone, e.g. via Bluetooth, to make the connection More generally, however, it is to be understood that the term “client” is used herein merely to denote the person monitored by the TIA device to detect a possible TIA episode experienced by the client accordingly, the TIA detection device may optionally be a single-purpose device designed to detect TIAs but not providing other services such as PERS monitoring.

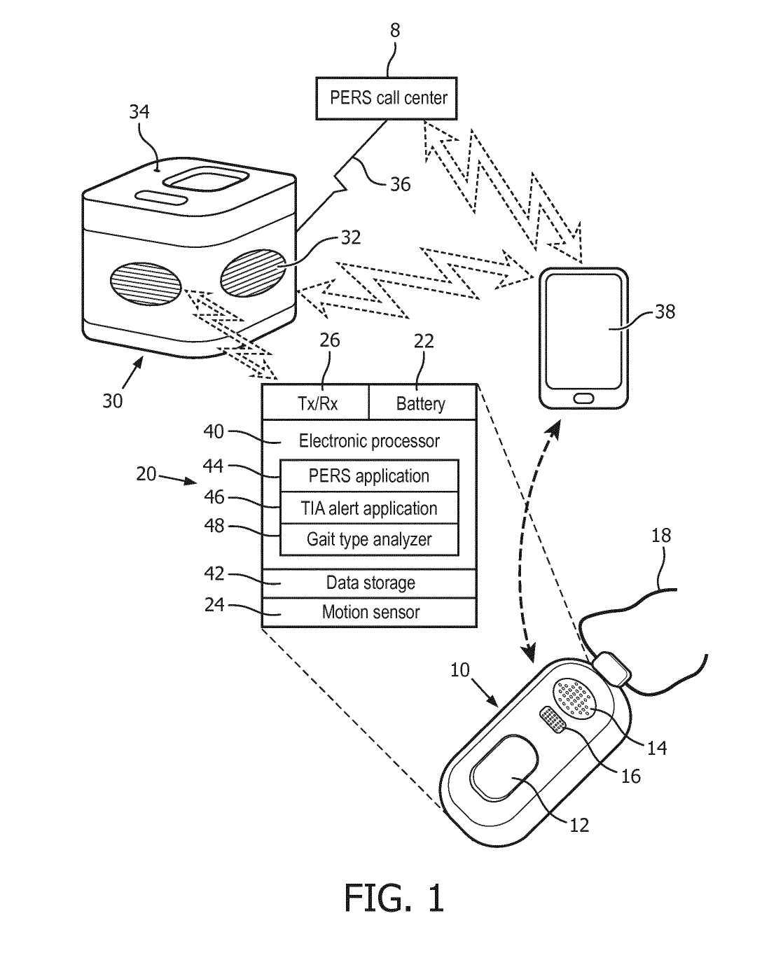

With reference to FIG. 1, an illustrative TIA alerting device operates in the context of a Personal Emergency Response Service (PERS) in which a PERS call center 8 can be reached by the client using a personal help button (PHB) device 10 worn by the client. The PERS call center 8 is staffed by agents each having an electronic work station typically including a computer on which a client's profile may be displayed and telecommunication equipment such as a headset via which the agent can converse with a client (details not shown). The client carries or wears the PHB device 10 which includes a call button 12, a speaker 14, and a microphone 16. The illustrative PHB device 10 is a pendant that is worn around the neck via a necklace 18 (shown in part). More generally, the PHB device is a unitary device that can have any suitable wearable form factor, such as the illustrative necklace-worn pendant, or a bracelet or wristband mount, a waist-mounted unit attached to a belt, or so forth. The PHB device 10 includes simple and effective mechanism such as the illustrative push button 12 for triggering a call to the PERS call center 8. While the illustrative (preferably large) push button 12 is a convenient call trigger mechanism, other call trigger mechanisms are contemplated, such as a voice-activated trigger mechanism.

An inset 20 diagrammatically indicates other functional components of the PHB device 10. A battery 22 powers the PHB device 10 to provide portability. The PHB device 10 further includes a motion sensor 24, such as (by way of non-limiting example) an accelerometer, a magnetometer, an inertial measurement unit (IMU) typically including an accelerometer and one or both of a gyroscope and/or magnetometer as a unitary assembly, or a pressure transducer. In some PHB devices the motion sensor 24 is provided for use as a “fall detector”, that is, to detect the client falling to the floor. Some examples of a PHB device including a motion sensor configured to provide fall detection are described in Peng et al., “Fall Detection System”, U.S. Pub. No. 2011/0162433 A1. In embodiments disclosed herein, the motion sensor 24 is used (in addition to or instead of its known use for fall detection) as a sensor for detecting motion due to the client's gait and classifying the gait type of the gait of the client. A radio transceiver 26 provides communication between the PHB device 10 and a base station 30 that has a speakerphone, i.e. a speaker 32 and a microphone 34, and that is communicatively connected with the PERS call center 8 via a wired telephone landline 36 or another communication link (e.g. a wireless cellular link, RF cable also carrying cable television signals, or so forth). The level of communication provided by the radio transceiver 26 may vary depending upon the designed capabilities of the PERS system. In a limiting case, the transceiver is actually only a transmitter (with no reception capability) that is capable of sending a fixed tone in response to activation of the call button 12 to trigger the base station 30 to place an emergency call to the PERS call center 8, and is further capable of modulating the tone to communicate binary data so as to send information collected via the TIA questionnaire to the base station 30 for relay to the PERS call center 8 (or to a 911 operator or so forth). In a more advanced PERS implementation, the transceiver 26 has both transmit and receive capability and may, for example, enable transmission of the PERS call center agent voice signal to the PHB device 10 if the client wearing the PHB device 10 is out of hearing range of the base station 30. It is further noted that in some embodiments, the transceiver 26 may additionally or alternatively include cellular communication capability (e.g. 2G, 3G, or 4G cellular connection) so that the PHB device 10 can place an emergency call to the PERS call center 8 directly, without the intermediary of the base station 30. Alternatively, the PHB device 10 can pair with a cellular telephone 38, for example via a wireless Bluetooth connection, to place the emergency call directly to the PERS call center 8 or via the base station 30. Such arrangements can, for example, provide PERS monitoring and connectivity for the mobile client who occasionally leaves the residence.

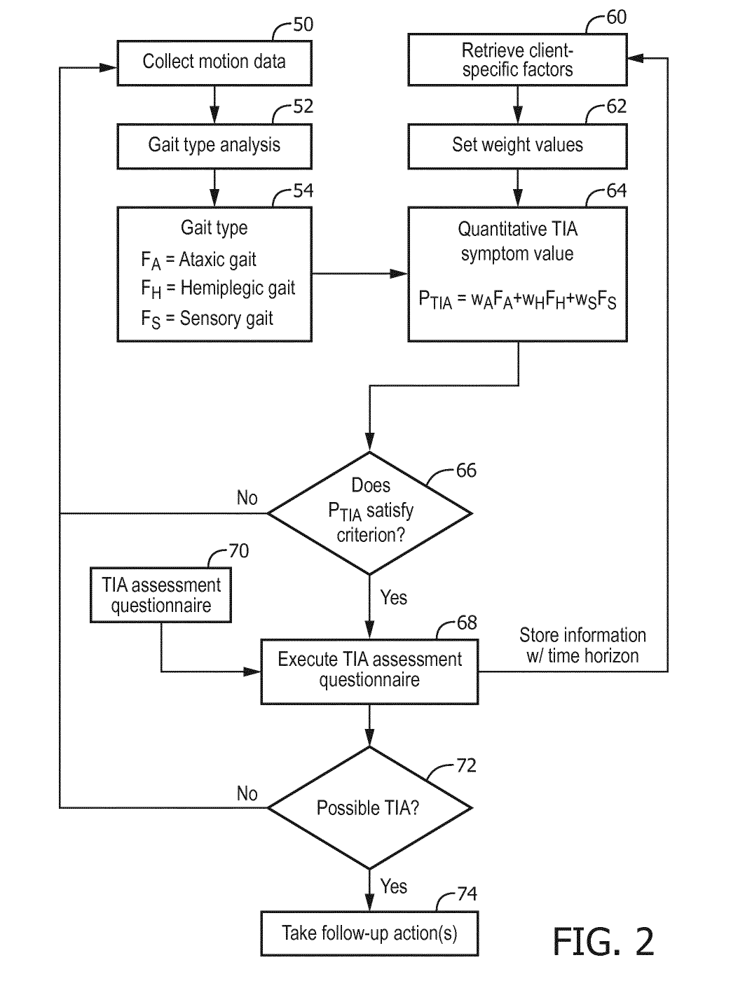

With returning reference to the inset 20, the PHB device 10 further includes an electronic processor 40 (e.g. a microprocessor or microcontroller) and electronic data storage 42 (e.g. flash memory, a solid state drive (SSD), or so forth). The electronic processor 40 is programmed by suitable application programs/software to execute a PERS application 44 that provides PERS alerting functionality such as: detecting activation of the call button 12 and in response causing the transceiver 26 to emit the appropriate trigger signal to trigger the base station 30 to place an emergency call to the PERS call center 8; monitoring motion sensor data collected by the motion sensor 24 to detect a fall event and responding appropriately (e.g. as described in Peng et al., “Fall Detection System”, U.S. Pub. No. 2011/0162433 A1); and/or so forth. The electronic processor 40 is further programmed by suitable application programs/software to execute a TIA alerting application 46 that implements the TIA alerting approaches disclosed herein. To this end, the TIA alerting application 46 includes or accesses a gait analyzer application 48 that processes motion data collected by the motion sensor 24 to detect a gait type that may suggest a possible TIA, such as an ataxic gait, a hemiplegic gait, or a sensory gait. Upon detection of such a TIA-suggestive gait, the TIA alerting application 46 proceeds to present questions of a TIA assessment questionnaire (see FIG. 2) via the speaker 14 and to receive verbal responses to the questions from the client via the microphone 16. The questionnaire may be stored in the storage 42, or downloaded in real-time via the transceiver 26. As described in more detail with later reference to FIG. 2, depending upon the gait type information collected by the motion sensor 24 and gait analyzer 48, together with the answers provided to the questions of the TIA assessment questionnaire, a decision is made whether to take follow-up action in response to a possible TIA. The follow-up action may, for example, entail invoking the PERS application 44 to transmit the trigger signal to the base station 30 to place the client into telephonic communication with the PERS call center 8, and/or to place a 911 call. Optionally, the follow-up actions may also include transmitting the questionnaire responses to the PERS call center 8 via the transceiver 26 and base station 30. In executing the foregoing activities, the electronic processor 40 suitably utilizes the data storage 42, for example by reading software stored in the data storage 42 that, when executed by the processor 40, implements the PERS application 44, TIA alerting application 46, and gait type analysis application 48. The electronic processor 40 may further write data to the data storage 42, such as writing motion sensor data collected by the motion sensor 24, and/or writing responses to the questions of the TIA assessment questionnaire (e.g., writing the recorded verbal response audio content in MP3 or another audio format, and/or in textual form after performing speech recognition on the received verbal response).

FIG. 1 diagrammatically illustrates selected internal components of the PHB device 10 in the inset 20. It will be appreciated that these various components may be variously integrally formed and/or mounted separately or as combined units in the housing of the mobile help button device 10. For example, as a hybrid integrated circuit, monolithic integrated circuit, or so forth.

With reference to FIG. 2, some illustrative examples are described of TIA alerting methods suitably performed by the TIA alerting device of FIG. 1. In an operation 50, motion sensor data are collected by the motion sensor 24. In an operation 52, the gait type analyzer 48 analyzes the motion sensor data to classify the client's gait as a gait type 54. Of interest as a symptom of a possible TIA event are ataxic, hemiplegic, or sensory gait types. An ataxic gait is characterized by unsteadiness or lack of coordination. A TIA episode can lead to an ataxic gait due to compromised motor nerve system function and/or impaired cognitive state leading to degraded conscious mobility control. A hemiplegic gait is characterized by strong left-right asymmetry and/or circumduction. A TIA episode can lead to a hemiplegic gait if it produces a higher degree of motor nerve system impairment on one side of the body compared with the other this is a common occurrence in a TIA since it may affect only one side of the brain to impair motor control on the opposite body side, while the unaffected side of the brain has little or no motor control impairment. A sensory gait, also called “stomping gait”, is characterized by heavy footfalls due to compromised propioreception in the foot. A TIA episode can lead to sensory gait due to impairment of propioreception in a foot and/or lower leg extremity.

The gait type analyzer 48 suitably operates as follows. Motion sensor data collected by the accelerometer or other motion sensor 24 over time form a data stream. Gait assessment by analysis of a motion sensor data stream using empirically learned classifiers or the like are known, see e.g. Wang et al., “A Method of Walking Parameters Estimation Via a 3-axis Accelerometer”, 2013 Int'l. Conf. on Orange Technologies (ICOT), pp. 298-301 (Mar. 12-16, 2013); Patterson et al., “A Novel Approach for Assessing Gait using Foot-Mounted Accelerometers”, 2011 5^{th} Intl. Conf. on Pervasive Computing Technologies for Healthcare (PervasiveHealth) and Workshops, pp. 218-221 (2011) (also assesses gait motions such as the flat-foot, heel/toe off, and leg swing phases); Chung et al., “Gait Analysis for Patients with Alzheimer's Disease Using a Triaxial Accelerometer”, 2012 IEEE Int'l. Symp. on Circuits and Systems, pp. 1323-26 (May 20-23, 2012); Yang et al., “iGAIT: An interactive accelerometer based gait analysis system”, Computer Methods and Programs in Biomedicine vol. 108 pp. 715-723 (2012) (detects gait features such as symmetry and stride regularity); Tumkur et al., “Modeling Human Walking for Step Detection and Stride Determination by 3-Axis Accelerometer Readings in Pedometer”, 2012 Fourth Intl. Conf. on Computational Intelligence, Modelling and Simulation (2012). Using such approaches, the gait type analyzer 48 outputs the gait type 54 as a value F_{A} representing the ataxic gait type component (e.g., based on stride length and stride regularity features extracted from the motion sensor data stream), a value F_{H} representing the hemiplegic gait type component (e.g., based on gait symmetry features), and a value F_{S} representing the sensory gait type component (e.g., based on features characterizing the flat-foot and heel/toe off gait phases). The gait type classifiers outputting the F_{A}, F_{H}, and F_{S} gait type components may be trained, for example, on motion sensor data examples collected from patients with normal gait (negative examples) and patients with the relevant gait type (positive examples, e.g. collected from patients diagnosed with the gait type due to a prior stroke). Moreover, since a TIA is likely to produce as a symptom a transient increased value of one or more of these components as compared with the client's gait prior to the TIA episode, in some embodiments the motion sensor data of the client are collected continuously or at certain intervals (e.g. 2 min/day) and analyzed to generate reference values for F_{A}, F_{H}, and F_{S}, and the gait type component values that are used for TIA detection are normalized or otherwise scaled by these reference values. For example, if the reference is collected over the last month, then F_{A}=F_{A,current}/|F_{A}|ₘₒₙₜₕ (and analogously for F_{H} and F_{S}).

With continuing reference to FIG. 2 and with further reference to Table 1, for improved accuracy the initial phase of detecting a TIA-symptomatic gait type may be adjusted based on one or more client-specific factors, such as one or more of those listed in Table 1. To this end, in an operation 60 one or more client-specific factors are retrieved from the data storage 42. Table 1 provides some examples. PHB device placement can impact the sensitivity of the motion sensor 24 to various types of gait. For example, an accelerometer disposed on a foot may be more effective for detecting a stomping gait compared with one disposed on the torso; whereas, a pendant-placed motion sensor may be more effective in detecting lateral swinging motion characteristic of a hemiplegic gait type. (In this regard, it is noted that while in the illustrative embodiment the motion sensor 24 is integral with the PHB device 10, in a variant embodiment the motion sensor may include one or more accelerometers or other motion sensors separate from the PHB device and mounted at optimal locations for detecting the various TIA-symptomatic gait types. In such embodiments, the separate motion sensors may communicate data to the PHB device via wireless connectivity such as Bluetooth™.) The “mobility history”, various “chronic condition” (e.g. vascular disease), “Smoker”, and like factors captures correlations of these conditions with increased likelihood of TIA. The “Doctor assessment of client” factor allows for tuning of the sensitivity of the TIA detection based on doctor assessment of the client's proneness to having a TIA.

TABLE 1
Examples factors for weighting TIA likelihood

| Factor | Significance |
|---|---|
| PHB device placement | Affects accuracy of gait asymmetry and |
| | stomping gait detection |
| Mobility history (over time | Reduced mobility over a recent time |
| horizon) | horizon may indicate increased TIA |
| | likelihood |
| Chronic [condition] | Depending upon [condition], may |
| | indicate increased TIA likelihood |
| Smoker | Increased TIA likelihood |
| Past stroke or diagnosed TIA(s) | Increased TIA likelihood |
| Age | Higher age increases TIA likelihood |
| Weight (ort BMI) | Higher BMI increases TIA likelihood |
| Doctor assessment of client | Doctor may indicate concern for TIA - |
| | use to increase weights |

In an operation 62, the patient-specific factors are used to set adjustable weights w_{A}, w_{H}, and w_{S} for weighting the F_{A}, F_{H}, and F_{S} gait type components, respectively, to form a quantitative TIA symptom value 64 according to P_{TIA}=w_{A}F_{A}+w_{H}F_{H}+w_{S}F_{S}. The value P_{TIA} can be thought of as a “probability” (though not necessarily normalized, i.e. not necessarily bound to be in the range [0,1]) that the client is exhibiting a TIA-symptomatic gait type. If so, then in an operation 66 process flow of the TIA alert application 46 passes to the second phase in which a TIA assessment questionnaire 70 is executed 68. If not, then flow passes back to operation 50 to continue monitoring client gait.

Another factor that may be taken into account in computing the quantitative TIA symptom value is the suddenness of onset of the TIA-symptomatic gait type. This takes into account that a TIA episode is an abrupt event, so that the resulting change in gait type is typically rapid (on the order of a few minutes or less).

It is noted that the foregoing are merely illustrative embodiments of the gait analysis phase. The F_{A}, F_{H}, and F_{S} gait type components are merely illustrative examples, and a sub-set of these gait types, and/or other gait type quantifications, can be employed as the TIA-suggestive gait symptom for triggering the TIA assessment questionnaire.

Moreover, while gait type is used herein as the TIA-suggestive symptom for triggering the TIA assessment questionnaire, (an)other TIA-suggestive symptom(s) may be employed depending upon the patient data being collected. For example, if the client is wearing eyewear (e.g. “smart” glasses) that monitor the client's eyeball motions, then certain eyeball movement patterns that correlate with TIA episodes could be used as the TIA-suggestive symptom for triggering the TIA assessment questionnaire execution.

With continuing reference to FIG. 2 and with further reference to Table 2, if at the operation 66 it is determined that the monitored TIA symptom (namely the TIA-symptomatic gait type in the illustrative examples) is observed in the subject motion sensor data, then in an operation 68 a TIA assessment questionnaire 70 is executed using the speaker 14 and microphone 16. Table 2 presents some non-limiting illustrative questions that may be included in the TIA assessment questionnaire 70, along with the significance of the response. Each question may be presented to the client by playback of a recording of the question being read by a human narrator (e.g, stored in the data storage 42); alternatively, each question may be articulated using speech synthesis technology. Responses to the electronically presented questions are detected by the microphone 16, and are interpreted using speech recognition software. The questions are preferably designed to be answered by “Yes” or “No”, which simplifies and improves accuracy of the speech recognition and is more likely to be answered accurately by the client who may be cognitively limited by a TIA or by an existing chronic condition such as dementia. However, it is also contemplated to employ questions requiring more complex answers.

TABLE 2
Example of a TIA Assessment Questionnaire

| Question | Significance |
|---|---|
| Are you feeling dizzy or | Yes - higher TIA probability |
| unsteady? | |
| Is your vision blurry, or do you | Yes - higher TIA probability |
| have any blindness? | |
| Do you feel tingling or numbness | Yes - higher TIA probability |
| in your face, arms, or legs? | |
| Do you have a bad headache? | Yes - higher TIA probability |
| Have you had an alcoholic drink | Yes - lower TIA probability |
| recently? | |
| Please repeat the following | Store audio recording of response to |
| sentence: “I like good weather, | provide to doctor to assess for |
| yes. I do not like rain, no.” | slurring; optionally perform |
| | automated classification of slurring |
| All questions - Speech | Elevated recognition uncertainty - |
| recognition uncertainty level | possible slurring - higher TIA |
| | probability |
| One or more questions - | Yes - higher TIA probability |
| unintelligible response | |
| Any question - no response | Take follow-up action(s) |

In some embodiments, the audio of all responses to the TIA assessment are recorded (with informed patient consent obtained when setting up the TIA alerting device), as review of the spoken answers by a doctor may be useful in diagnosing a TIA. For example, the patient's articulation may evidence slurring (or lack thereof), and/or delayed or confused responses may indicate cognitive difficulty (possibly due to a TIA). Additionally, if the speech recognition is unable to discern the answer it is possible the doctor may be able to understand the answer when listening to playback of the recording. In this regard, it is contemplated to have a dedicated slur test question intended to elicit a more complex spoken answer to provide for better assessment of any slurring in the recording playback. The illustrative slur test question of Table 2 is the question: “Please repeat the following sentence: ‘I like good weather, yes. I do not like rain, no.’” More generally, the slur test sentence may be chosen to include phrases, in the natural language spoken by the client, that are designed or chosen by speech therapy experts as being effective for detecting slurring or other types of speech difficulty. To provide a baseline for comparison, the TIA alerting device may optionally be programmed to request that the client respond to the slur test question on a regular basis, e.g. once a month, and the last few recordings of the response to the slur test question may be stored in device memory 42 to provide the baseline for comparison. The illustrative slur test sentence also includes the words “yes” and “no”—this is optional, but if included and the slur test question is posed on a monthly or other basis for baseline purposes then the articulated “yes” and “no” words in the response can be used for update training of the speech recognition software in recognizing these key responses.

In some embodiments, the set of questions making up the TIA assessment questionnaire 70 can be chosen for the specific patient during setup of the TIA alerting device. For example, a list of all available questions with checkboxes can be provided to the doctor or other expert setting up the device, and the doctor checks those questions to pose during execution 68 of the TIA assessment questionnaire 70. By way of illustration, the TIA assessment questionnaire example of Table 2 includes the question “Have you had an alcoholic drink recently?” which is intended to provide information for distinguishing inebriation from a TIA. However, some patients might be offended by this question, or the doctor may decide that even if inebriation is generating TIA-mimicking symptoms this should nonetheless produce a TIA alert (perhaps so that the patient is treated for inebriation). Thus, the doctor may or may not elect to include this question for a particular patient based on the doctor's assessment of its appropriateness.

The illustrative TIA assessment questionnaire 70 of Table 2 assumes the TIA alerting device 10 has audio communication capability, that is, the ability to play back or synthesize spoken questions directed to the client and a microphone for recording spoken responses provided by the client. Other communication pathways are contemplated, for example if the client is unable to hear and/or speak intelligibly due to some medical condition then an alternative mode of communication could be provided, such as a display screen.

As another contemplated variation, if the TIA alerting device includes still photo and/or video capability (for example, the base station having a video display and a video camera for recording video of the client, or leveraging a camera of cellphone 38), then the TIA assessment questionnaire may optionally include additional questions having responses that are capable of being recorded by the video camera. For example, a possible question could be “Please look at the dot on the display and smile”. The response would be the video recording of the patient's face, and facial recognition software may then be applied to identify and classify the patient's face as to whether it is exhibiting face droop (which can be a symptom of TIA). Similarly, the patient could be asked to raise both arms, and the video camera used to detect whether an arm hangs down (again a possible symptom of TIA). In configurable questionnaire embodiments these questions may be selected for inclusion in the TIA alerting device of a specific patient only if the requisite video hardware is to be installed in the client's residence (and perhaps only in homebound situations where the client is likely to be in close proximity to that video hardware during a possible TIA).

As noted in the example of Table 2, in addition to the semantic content of the client's responses (e.g., the “Yes” or “No” answers in the example of Table 2), other information that may be probative for diagnosing a TIA episode may be extracted from the responses to the questionnaire 70. For example, if the speech recognition software generates a recognition confidence level for detected “Yes” and “No” answers, then this confidence level can be used as further information, since a low confidence in the answer detections may be indicative of slurred speech which is a possible TIA symptom. Likewise, unintelligible answers (those for which no semantic content is extracted by the speech recognition) are an extreme case of maximal speech recognition uncertainty, and may be indicative of severe slurring. If the client provides no response at all to one or more questions, this may be due to the client being unconscious or in a non-responsive (or intermittently responsive) cognitive state in this case appropriate follow-up action may be taken such as placing a call to the PERS center and/or calling “911” or another emergency response telephone number to summon an ambulance.

The specific content of the TIA assessment questionnaire may optionally, as already mentioned, be chosen for the specific client and/or TIA alerting device installation hardware (e.g. whether the video component is installed). Additionally, as the illustrative TIA alerting device 10 is in communication with the PERS center 8 via the base station 30, it is possible to download questionnaire updates as they become available, so that the questionnaire 70 embodies the latest information on the most effective tools for identifying a possible TIA.

With continuing reference to FIG. 2, at a decision operation 72 a TIA assessment is determined based at least on the spoken responses to the questions of the TIA assessment questionnaire 70. The specific assessment may be chosen based on the questions of the questionnaire 70, and optionally on other factors such as the client-specific factors 60. For example, referring to the illustrative questionnaire of Table 2, it may be appropriate to provide a positive TIA assessment (that is, to conclude the client has undergone a possible TIA episode calling for medical evaluation) if any one of certain critical questions are answered with “Yes”, such as the question “Is your vision blurry, or do you have any blindness?” This is based on the elicited symptom (in this case, blurry vision or partial vision loss) is a strong indicator of a possible TIA episode. On the other hand, some questions elicit less strong indicators, such as the question “Are you feeling dizzy or unsteady?” for which an affirmative response could be due to a TIA but also could be due to other causes such as a low blood pressure episode, standing quickly, or so forth. For such “less probative” questions, two or more affirmative answers may be required to reach an affirmative TIA assessment calling for medical evaluation. In some embodiments, the TIA assessment is further based on the quantitative TIA symptom value 64, e.g. if the client is exhibiting a strongly hemiplegic gait type then this may bias the decision 72 toward an affirmative assessment of a possible TIA. Similarly, various client-specific factors 60 may be incorporated into the decision, e.g. if the client's doctor has indicated a high likelihood of TIA then this may bias toward an affirmative TIA assessment.

It will be appreciated that since the number of questions in the TIA assessment questionnaire 70 is low (given the time constraints for assessing a possible TIA episode, it is undesirable for the questionnaire to include dozens of questions) and most or all questions may preferably be designed to elicit binary (yes or no) answers, it is possible to manually construct a TIA assessment algorithm. For example, the questionnaire of Table 2 includes only five binary-response questions (not counting the slurring question), so that there are only 2⁵=32 possible answer combinations. Thus, it is feasible to have a medical expert evaluate each of these thirty-two possible response combinations individually and pre-assign a risk score.

If the TIA assessment determined in the operation 72 is affirmative, that is, if it is determined that the client has undergone a possible TIA episode calling for medical evaluation, then in an operation 74 appropriate follow-up action(s) are taken. Some contemplated follow-up actions include: placing a call to the PERS call center 8 to place the client into telephonic communication with a PERS agent; placing a call to 911 or another emergency number to summon an ambulance; instructing the client to sit down via the speaker 14; various combinations of these actions; or so forth. Depending upon the communication capability provided by the transceiver 26, the follow-up actions could include transmitting the client's spoken response to the slurring test question to the PERS agent and/or to the 911 operator. Alternatively, this response may be stored in the memory 42 for later offloading by the client's doctor for review during a follow-up office visit. The follow-up action(s) may also be dependent upon the client responses to the questionnaire 70—for example, if the client reports dizziness and tingling of the limbs, but no vision impairment, then the follow-up response may be limited to placing a call to the PERS call center 8; whereas, if the client reports partial blindness, or is entirely non-responsive, then a call to 911 to summon an ambulance may be appropriate. Where the follow-up action includes placing a call, e.g. to 911 and/or the PERS call center 8, the call may be an audio call and/or may be a data transmission. For example, the call could include transmission of GPS coordinates or other location information obtained from the cellular telephone 38 (if carried by the client and electronically accessible) or obtained from a GPS unit built into the PHB device 10 (component not shown).

As further indicated in FIG. 2, in some embodiments information obtained by execution 68 of the TIA assessment questionnaire 70 may additionally be fed back to update the client-specific factors 60. For example, although an affirmative response to the question “Do you have a bad headache?” might not, by itself, be sufficient to generate a positive response at the possible TIA decision 72, this information might be used to increase one or more of the weights so as to bias toward detection of a TIA-symptomatic gait type at some subsequent iteration of the operation 64. In the illustrative example, such feedback has some time horizon so that the weight(s) increase is temporary.

In the illustrative embodiment, the TIA assessment questionnaire 70 is executed 68 by the PHB device 10 using its built-in speaker 14 and microphone 16. In another contemplated embodiment, the TIA assessment questionnaire is executed by the base station 30 using its speaker 32 and microphone 34. In this alternative embodiment, the motion sensor data analysis (gait type analysis 52 and quantitative TIA symptom value calculation 60, 62, 64) may be performed by the PHB device 10, or the motion sensor data may be transmitted to the base station via the transceiver 26 and the sensor data analysis performed at the base station (so that operations 60, 62, 64 are also performed by the base station 30).

The illustrative embodiments have been described with reference to detecting and issuing an alert of a transient ischemic attack (TIA) episode, which generally does not produce lasting neurological damage or widespread tissue death. The disclosed TIA alerting device is well-suited for detecting a TIA because it is triggered by detection of a possible TIA symptom (gait type indicative of TIA in the illustrative examples) and operates to detect a possible TIA on a time frame of a few minutes, which is within the time frame of the transient symptoms of a typical TIA. It will be appreciated that the TIA alerting device can also operate to detect a true stroke, that is, a stroke that produces lasting neurological damage and/or widespread tissue death. If the stroke is sufficiently mild that the stroke victim retains effective cognition, then the TIA alerting device operates to detect the mild stroke in substantially the same way as the detection of a possible TIA as described herein. If the stroke is more serious such that the stroke victim becomes non-responsive and/or falls down (if initially in a standing position), then as previously described this will result in failure to respond to questions of the questionnaire leading to appropriate follow-up action (typically placing a call to the PERS center and/or to 911 or another emergency response number in accordance with a PERS nonresponsive client protocol and/or a PERS “fall” protocol initiated by detection of the client falling).

As already noted, the purpose of the TIA alerting device is not to provide a clinical diagnosis of a TIA, but rather to provide an early alert of a possible TIA, with the optional secondary purpose of collecting data to aid later diagnosis such as recording the client's speech for slur assessment. The disclosed TIA alerting device is useful to detect the TIA before its transient symptoms dissipate. In the case of a true stroke, symptoms are likely to persist since there is permanent neurological and/or tissue damage. However, in the case of a true stroke, rapid intervention is a key factor affecting survival likelihood and the extent of achievable rehabilitation. For example, rapid intervention can remove the vascular blockage (in the case of an ischemic stroke) or remediate hemorrhaging (in the case of a hemorrhagic stroke), thereby limiting the consequent neurological and/or tissue damage. Thus, regardless of whether the underlying condition is a transient TIA or a true stroke, the disclosed TIA alerting devices are operative to provide advantageous rapid detection and early alerting that is likely to benefit the client in terms of disease prevention and/or rehabilitation.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A transient ischemic attack (TIA) alerting device comprising:
a motion sensor configured to attach to or be worn by a subject; and
a wearable electronic data processing device including a speaker, a microphone, a radio transmitter, and an electronic processor programmed to perform a TIA alerting method including:
detecting a TIA-symptomatic gait type by processing subject motion data acquired by the motion sensor, wherein the detected TIA-symptomatic gait type comprises at least one of: an ataxic gait, a hemiplegic gait, and a sensory gait;
responsive to detecting the TIA-symptomatic gait type, executing a TIA assessment by generating a set of questions, outputting the set of questions using the speaker, and receiving spoken responses using the microphone;
determining a TIA assessment response based at least on the spoken responses to the set of questions of the TIA assessment; and
generating a TIA alert radio signal configured to be transmitted via the radio transmitter, wherein the TIA alert radio signal is conditional on the TIA assessment indicating a possible TIA.

2. (canceled)

3. The TIA alerting device of claim 1 wherein the TIA-symptomatic gait type includes an ataxic gait detected based on stride length and stride regularity features generated from the motion sensor data.

4. The TIA alerting device of claim 1 wherein the TIA-symptomatic gait type includes a hemiplegic gait detected based on gait symmetry features generated from the motion sensor data.

5. The TIA alerting device of claim 1 wherein the TIA-symptomatic gait type includes a sensory gait detected based on features generated from flat-foot and heel/toe off stride phase portions of the motion sensor data.

6. The TIA alerting device of claim 1 wherein the motion sensor comprises an accelerometer.

7. The TIA alerting device of claim 1 wherein the TIA alert radio signal is transmitted to
a base station configured to generate an emergency call in response to receiving the TIA alert radio signal.

8. The TIA alerting device of claim 1 wherein the radio transmitter comprises a cellular telephone transmitter and the TIA alert radio signal comprises a cellular call.

9. The TIA alerting device of claim 1 wherein the wearable electronic data processing device further includes a call button, wherein activation of the call button causes the radio transmitter to output an emergency alert signal.

10. The TIA alerting device of claim 1 wherein the motion sensor is integrated with the wearable electronic data processing device.

11. The TIA alerting device of claim 1 wherein the wearable electronic data processing device further includes an electronic data storage and the electronic processor is programmed to store at least one spoken response to at least one question of the TIA assessment questionnaire in the electronic data storage.

12. A transient ischemic attack (TIA) alerting device comprising:
a physiological sensor configured to attach to or be worn by a subject; and
an electronic data processing device including a speaker, a microphone, and an electronic processor programmed to perform a TIA alerting method including:
detecting a TIA symptom indicative of at least one of: an ataxic gait, a hemiplegic gait, and a sensory gait, by processing physiological data acquired by the physiological sensor;
responsive to detecting the TIA symptom, executing a TIA assessment questionnaire by outputting a set of questions using the speaker and receiving spoken responses using the microphone;
determining a TIA assessment response based at least on the spoken responses to the set of questions of the TIA assessment questionnaire; and
generating a TIA alert signal configured to be transmitted via a transmitter, wherein the TIA alert signal is conditional on the TIA assessment indicating a possible TIA.

13. The TIA alerting device of claim 12 wherein the physiological sensor is a motion sensor configured to detect the TIA symptom comprising a TIA-symptomatic gait type by processing motion sensor data acquired by the motion sensor.

14. (canceled)

15. The TIA alerting device of claim 13 wherein the TIA-symptomatic gait type comprises at least one of:
an ataxic gait detected based on stride length and stride regularity features generated from the motion sensor data,
a hemiplegic gait detected based on gait symmetry features generated from the motion sensor data, and
a sensory gait detected based on features generated from flat-foot and heel/toe off stride phase portions of the motion sensor data.

16. The TIA alerting device of claim 12 wherein the electronic data processing device comprises a wearable component configured to attach to or be worn by the subject, the wearable component including the speaker, the microphone, and a radio transmitter that outputs the TIA alert signal comprising a radio transmission signal conditional on the TIA assessment indicating a possible TIA.

17. The TIA alerting device of claim 16 wherein the electronic data processing device the TIA alert radio signal is transmitted to a base station configured to generate a further TIA alert signal comprising a telephonic emergency call in response to receiving the radio transmission signal.

18. The TIA alerting device of claim 16 wherein the radio transmitter comprises a cellular telephone transmitter that outputs the radio transmission signal comprising a cellular telephone call conditional on the TIA assessment indicating a possible TIA.

19. The TIA alerting device of claim 12 wherein the electronic data processing device comprises a speaker and the microphone, and wherein the electronic data processing device is configured to the TIA assessment to determine the TIA assessment, and output the TIA alert signal conditional on the TIA assessment indicating a possible TIA.

20. A transient ischemic attack (TIA) alerting method comprising:
detecting a TIA-symptomatic gait type by processing subject motion data acquired by a motion sensor, wherein the detected TIA-symptomatic gait type comprises at least one of: an ataxic gait, a hemiplegic gait, and a sensory gait;
responsive to detecting the TIA-symptomatic gait type, executing a TIA assessment by electronically generating a set of questions, out putting the set of questions using a speaker and electronically receiving spoken responses using a microphone;
determining a TIA assessment response based at least on the electronically received spoken responses to the electronically outputted set of questions of the TIA assessment; and
generating a TIA alert radio signal configured to be transmitted via a radio transmitter, wherein the TIA alert radio signal is conditional on the TIA assessment indicating a possible TIA.

21. The TIA alerting method of claim 20 wherein detecting the TIA-symptomatic gait type comprises at least one of: detecting an ataxic gait based on stride length and stride regularity extracted from the subject motion data, detecting a hemiplegic gait detected based on gait symmetry extracted from the subject motion data, and detecting a sensory gait detected based on flat-foot and heel/toe off stride phases extracted from the subject motion data.
